(19) United States
(12) Patent Application Publication
(10) Pub. No.: US 2006/0183766 A1
(43) Pub. Date: Aug. 17, 2006

(54) Orally bioavailable CCI-779 formulations

(75) Inventors: Joseph P. Boni, Wayne, PA (US); Muhammad Ashraf, Elmwood Park, NJ (US); Eric J. Benjamin, Jamestown, NC (US)

Correspondence Address: HOWSON AND HOWSON;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 11/352,726

(22) Filed: Feb. 13, 2006

Related U.S. Application Data
(60) Provisional application No. 60/652,889, filed on Feb. 15, 2005.

(51) Int. Cl. A61K 31/4745

(52) U.S. Cl. 514/291

(57) ABSTRACT

A CCI-779 oral dosage form is provided in which, after oral administration to a subject, the CCI-779 has a whole blood peak concentration (Cₘₐₓ) of 5.4±1.8 ng/mL and an area under the curve (AUC) of about 66±about 22 ng-hr/ml and the sirolimus has a Cₘₐₓ of 18.7±9.6 ng/mL and an AUC of about 600±about 228 ng-hr/ml, for a 25 mg unit dose of CCI-779. Another CCI-779 oral dosage form is provided which, after oral administration thereof to a subject, the CCI-779 has a Cₘₐₓ of 5.7±1.7 ng/mL and an AUC of about 60±about 20 ng-hr/ml and the sirolimus has a Cₘₐₓ of 17.1±8.1 ng/mL and an AUC of about 548±about 187 ng-hr/ml in whole blood, for a 25 mg unit dose of CCI-779. Products containing these oral dosage forms, and methods of use thereof, are also described.

### CROSS-REFERENCE TO RELATED APPLICATIONS

This application claims the benefit under 35 USC 119(e) of prior U.S. Provisional Patent Application No. 60/652,889, filed Feb. 15, 2005.

### BACKGROUND OF THE INVENTION

Rapamycin 42-ester with 3-hydroxy-2-(hydroxymethyl)-2-methylpropionic acid (CCI-779) is an anticancer agent and is characterized by the following structure.

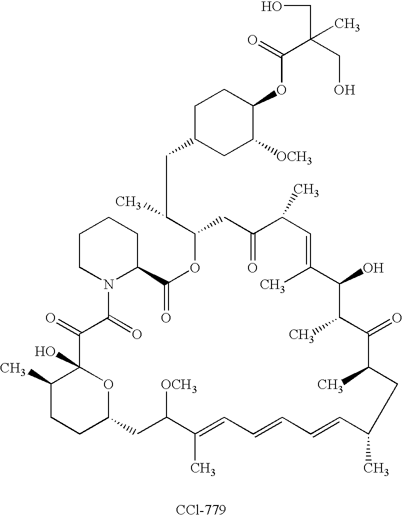

CCI-779 exhibits cytostatic, as opposed to cytotoxic properties, and may delay the progression of tumors or tumor recurrence. The mechanism of action of CCI-779 that results in the G1 to S phase block is novel for an anticancer drug. In vitro, CCI-779 has been shown to inhibit the growth of a number of histologically diverse tumor cells. Central nervous system (CNS) cancer, leukemia (T-cell), breast cancer, prostate cancer, and melanoma lines were among the most sensitive to CCI-779. The compound arrested cells in the G1 phase of the cell cycle.

CCI-779 has poor water solubility (less than 1 μg/ml) and high permeability (Log PC≧4.1 in 1-octanol/water system and P_{cff}=4−5×10⁻⁵ cm/sec obtained from in situ rat intestinal perfusion study using metoprolol tartrate as a marker) and is classified as class II compound according to BCS classification system. One obstacle towards the formulation of CCI-779 is its poor aqueous dissolution and low oral bioavailability. Additionally, CCI-779 exhibits aqueous instability and has shown its potential to undergo oxidation.

A CCI-779 formulation was developed that employed a wet granulation manufacturing process. See US Published Patent Application No. US-2004-0077677-A1. This process involved preparation of a hydroalcoholic granulation solution of CCI-779. Further, although the resulting tablets were stable and bioavailable, the preparation of the hydroalcoholic solution was very tedious. Further, CCI-779 was thermodynamically unstable, precipitating within one day after its preparation, requiring it to be used immediately after its preparation.

A bioavailable CCI-779 oral formulation which can be conveniently manufactured is desirable.

### SUMMARY OF THE INVENTION

The present invention provides a convenient and effective method to deliver therapeutic levels of CCI-779 to the patient.

In one aspect, the invention provides a composition comprising an effective amount of CCI-779 wherein, after oral administration thereof to a subject, the CCI-779 has a whole blood peak concentration (Cₘₐₓ) of 5.4±1.8 ng/mL and an area under the curve (AUC) of about 66±about 22 ng-hr/ml and the sirolimus has a Cₘₐₓ of 18.7±9.6 ng/mL and an AUC of about 600±about 228 ng-hr/ml, for a 25 mg unit dose of CCI-779. In a further aspect, the invention provides a composition wherein, after oral administration to a subject, the CCI-779 has a Tₘₐₓ of 2.0±1.8 hours. In one embodiment, the CCI-779 oral formulation comprises micronized CCI-779 in a high povidone-containing formulation.

In another aspect, the invention provides a composition comprising an effective amount of CCI-779 wherein, after oral administration thereof to a subject, the CCI-779 has a Cₘₐₓ of 5.7±1.7 ng/mL and an AUC of about 60±about 20 ng-hr/ml and the sirolimus has a Cₘₐₓ of 17.1±8.1 ng/mL and an AUC of about 548±about 187 ng-hr/ml in whole blood, for a 25 mg unit dose of CCI-779. In a further aspect, the invention provides a composition wherein, after oral administration to a subject, the CCI-779 has a Tₘₐₓ of 1.3±0.6 hours. In one embodiment, the CCI-779 oral formulation comprises micronized CCI-779 in a low povidone-containing formulation.

In another aspect, the invention provides a method of treating a subject with the compositions of the invention, and use of the compositions of the invention in preparing medicaments useful in the treatment of a subject.

The invention further provides kits and other products containing the compositions of the invention.

Other aspects and advantages of the invention will be readily apparent from the following detailed description of the invention.

### DETAILED DESCRIPTION OF THE INVENTION

The invention provides micronized CCI-779 compositions of the invention in an oral dosage form containing an effective dose of CCI-779 which having the pharmacokinetic profile described herein.

The invention further provides a method of achieving a bioavailability of CCI-779 in a subject, preferably human, upon administering orally a CCI-779 oral dosage form, such that an AUC and Cₘₐₓ in the ranges provided above is achieved. The invention also provides a method of treating a human by administering an effective dose of the CCI-779 compositions of the invention such that the AUC and Cₘₐₓ in the ranges provided above are achieved.

Advantageously, the compositions of the invention can be readily manufactured using micronized CCI-779. CCI-779 is micronized under nitrogen and conventional micronizing techniques, for example with a Trost or jet mill, applied to non-micronized CCI-779. The preparation of non-micronized CCI-779 is described in U.S. Pat. No. 5,362,718, which is hereby incorporated by reference. A regioselective preparation of non-micronized CCI-779 is described in U.S. Pat. No. 6,277,983, which is hereby incorporated by reference. However, the invention is not limited to the method by which the non-micronized CCI-779 is produced. Alternatively, CCI-779 can be purchased commercially (e.g., Wyeth). Micronized CCI-779 typically has a particle size of about 0.2 to about 30 microns, about 0.5 to 25 microns, or about 0.5 to 20 microns, as described above.

In one embodiment, the compositions of the invention contain micronized CCI-779 with a particle size range in which 10% are less than or equal to about 3 microns (μ), 50% are about 10 μ, and 90% are less than or equal to about 20μ as determined by Malvern method. In one embodiment, the micronized CCI-779 has a particle size range of 10% are less than or equal to about 2μ, 50% are about 5μ, and 90% are less than or equal to about 16μ as determined by Malvern method.

Suitably, the micronized CCI-779 is present in the composition of the invention in an amount from 0.1 % w/w to 50% w/w, based on the weight of an uncoated composition of the invention. This amount may be varied, depending upon the amount of micronized CCI-779 to be delivered to a patient. For example, an effective amount of micronized CCI-779 is generally in the range, e.g., about 0.1 to about 50 mg, about 10 mg to about 30 mg, or about 0.5 to about 2 mg micronized CCI-779. The desired therapeutic regimen can be taken into consideration when formulating a composition of the invention. For example, micronized CCI-779 can be in the range of 0.1% w/w to 10% w/w for an uncoated composition of the invention. In another example, micronized CCI-779 can be in the range of 5% w/w to 25% w/w based upon the weight of an uncoated unit dose. In yet another example, micronized CCI-779 can be in the range of 6% w/w to 8% w/w, 15% w/w to 40% w/w, or 20% w/w to 30% w/w based on the weight of an uncoated unit dose.

In addition to containing micronized CCI-779, the composition of the present invention can contain pharmaceutically acceptable additives and/or excipients. Typically, these additives are biologically inert and useful for manufacture of a dosing unit. The compositions of the invention may contain one or more filler/binder, disintegrant, a dissolution enhancer (including, e.g., a surfactant), glidant, and lubricant. In certain embodiments, the compositions further contain one or more antioxidants, chelating agents, or pH modifiers. Optionally, the antioxidant, chelating agent, and/or pH modifier may be micronized. Micronized additives and excipients are prepared using conventional techniques, as described.

Examples of pharmaceutically acceptable binders, fillers, and disintegrants include sucrose, lactose, magnesium stearate, gum acacia, cholesterol, tragacanth, stearic acid, gelatin, casein, lecithin (phosphatides), carboxymethylcellulose calcium, carboxymethylcellulose sodium, methylcellulose, hydroxyethylcellulose, hydroxypropylcellulose, hydroxypropylmethycellulose phthalate, noncrystalline cellulose, cetostearyl alcohol, cetyl alcohol, cetyl esters wax, dextrates, dextrin, lactose, dextrose, glyceryl monooleate, glyceryl monostearate, glyceryl palmitostearate, polyoxyethylene alkyl ethers, polyethylene glycols, polyoxyethylene castor oil derivatives, polyoxyethylene stearates, and polyvinyl alcohol, and the like.

In one embodiment, the disintegrant is croscarmellose sodium. Suitably, a composition of the invention contains a total of about 3% w/w to 8% w/w disintegrant, e.g., about 4% to about 6% w/w.

The binders and fillers can be selected from the group consisting of polyvinylpyrrolidone (povidone), lactose (including anhydrous lactose), and microcrystalline cellulose, and mixtures thereof. Suitably, a composition of the invention contains a total of about 75% w/w to 88% wlw binder/filler, or about 80% w/w to 82% w/w binder/filler, based on the weight of an uncoated composition. For example, a composition of the invention may contain, in addition to the micronized CCI-779 and other components, about a low amount of povidone, e.g. about 5 to 7% w/w, and more desirably, about 6% w/w, with the remainder of the filler in the uncoated composition being supplied by other components. In another example, a composition of the invention may contain a high amount of povidone, e.g., about 25 to 35% w/w, and more desirably, about 30 to 32% w/w povidone, with the remainder of the filler in the uncoated composition being supplied by other components. In yet another example, a composition of the invention contains a combination of lactose, preferably anhydrous lactose, and microcrystalline cellulose, optionally with povidone or another filler/binder. In such a composition (based on uncoated weight), anhydrous lactose is generally present in an amount of about 30% w/w to about 60% w/w, and more desirably, about 30% w/w, about 32% w/w, about 50% w/w, or about 55% w/w anhydrous lactose. Suitably, in such an uncoated composition, microcrystalline cellulose is present in an amount of about 15% w/w to about 30% w/w of the uncoated composition, and more desirably, about 16% w/w, about 23% w/w, about 25% w/w, about 28% w/w of the uncoated composition.

Dissolution enhancers may be included in the micronized CCI-779 composition (based on uncoated weight) of the invention. Preferably, one or more dissolution enhancers may optionally be present in the composition in an amount of from about 0.5% w/w to about 10% w/w, and preferably, from about 5% w/w to about 8% w/w, about 5.5%, about 6% w/w, or 6.5% w/w, based on the weight of an uncoated composition. Examples of dissolution enhancers include surfactants, chelating agents (e.g., EDTA), disintegrants, or combinations thereof.

In one embodiment, the surfactant is about 0.25 % w/w to about 10% w/w of an uncoated composition, and preferably, about 5% w/w to about 6.5% w/w. In one embodiment, the surfactant is selected from sodium lauryl sulfate (also known as sodium dodecyl sulfate). Other suitable surfactants are well known to those of skill in the art and can be selected including, without limitation, polysorbates including, e.g., polysorbate 80, Polaxamer 188™ surfactant, sodium lauryl sulfate (sodium dodecyl sulfate), salts of bile acids (taurocholate, glycocholate, cholate, deoxycholate, etc.) which may be combined with lecithin. Alternatively, ethoxylated vegetable oils, such as Cremophor EL, vitamin E tocopherol propylene glycol succinate (Vitamin E TGPS), polyoxyethylene-polyoxypropylene block copolymers, and poloxamers.

Acceptable antioxidants include, but are not limited to, citric acid, d,l-α-tocopherol, butylated hydroxyanisol (BHA), butylated hydroxytoluene (BHT), monothioglycerol, ascorbic acid, propyl gallate, and mixtures thereof. It is expected that the total amount of antioxidants in the formulations of this invention will be in concentrations ranging from 0.001% to 3% w/w, and preferably, about 0.01 w/w to about 1% w/w, and more preferably, about 0.02% w/w to 0.1% w/w, based on the weight of an uncoated composition. In one embodiment, the antioxidant is a combination of BHA and BHT, which may be in nonmicronized form or preferably, in micronized form.

Chelating agents and other materials capable of binding metal ions, such as ethylene diamine tetra acetic acid (EDTA) and its salts and hydrates (e.g., EDTA calcium disodium hydrous) are useful in the compositions of the invention. Typically, where present, a chelating agent is present in an amount less than 1% w/w, e.g., about 0.001% w/w to about 0.01% w/w, based on the weight of an uncoated composition. In one embodiment, the chelating agent is present in micronized form.

Acceptable pH modifying agents include, but are not limited to citric acid and salts thereof (e.g., sodium citrate), dilute HCl, and other mild acids or bases capable of buffering a solution containing CCI-779 to a pH of 4 to 6. Where present in a composition of the invention, such pH modifiers are present in an amount up to about 1% w/w, e.g., about 0.001% w/w to about 0.1% w/w, based on the weight of an uncoated composition. Optionally, the pH modifier, can be present in micronized form.

Other suitable components include lubricants and/or glidants. In one embodiment, the lubricant and the glidants can each be present in the composition of the invention in an amount of 0.01 wt % to about 1 wt %, about 0.1 wt % to about 2 wt %, or about 0.2 to about 0.5%, of an uncoated composition. In some embodiments, the lubricant and glidants are present in the composition in amounts of less than 1 wt % of an uncoated composition. An example of a suitable lubricant is magnesium stearate and an example of a suitable glidants is silicone dioxide.

Other suitable inert components of the formulation will be readily apparent to one of skill in the art.

The compositions of the invention are formed into a suitable dosing unit for oral delivery to a patient. Suitable dosing units include oral dosing units, such as a directly compressible tablet, a capsule, a powder and a suspension. These dosing units are readily prepared using the methods described herein and those known to those of skill in the art.

In one embodiment, a composition of the invention is prepared by dry mixing micronized CCI-779 with the other additives in a suitable mixer. The powder mix is then directly compressed into unit dose tablets.

Without limitation as to the method of preparation of a composition of the invention, an example of a suitable micronized CCI-779 formulation includes a low amount of povidone. The following weight percentages are based upon an uncoated composition of the invention.

| | | |
|---|---|---|
| CCI-779, Micronized | 6% | w/w; |
| Sodium Lauryl Sulfate | 6% | w/w; |
| Povidone | 6% | w/w; |
| Lactose Anhydrous | 50% | w/w; |
| Microcrystalline Cellulose | 25% | w/w; |
| Croscarmellose Sodium | 6% | w/w; |
| Glidant | 0.25% | w/w; and |
| Magnesium Stearate | 0.25% | w/w. |

Still a further example of a suitable micronized CCI-779 composition contains a high amount of povidone, with weight percentages based upon an uncoated composition of the invention:

| | | |
|---|---|---|
| Micronized CCI-779 | 6% | w/w; |
| Sodium Lauryl Sulfate | 6% | w/w; |
| Povidone | 31% | w/w; |
| Lactose Anhydrous | 34% | w/w; |
| Microcrystalline Cellulose | 16% | w/w; |
| Croscarmellose Sodium | 6% | w/w; |
| Glidant | 0.25% | w/w; and |
| Magnesium Stearate | 0.5% | w/w. |

Yet a further example of a suitable micronized CCI-779 dosing unit, with weight percentages based on total uncoated composition, is:

Yet another example of a suitable dosing unit, with weight percentages based on total uncoated composition, is:

| | | |
|---|---|---|
| CCI-779 (Micronized) | 6% | w/w; |
| Butylated Hydroxyanisole (Micronized) | 0.022% | w/w; |
| Butylated Hydroxytoluene (Micronized) | 0.05% | w/w; |
| EDTA Calcium Disodium, Hydrous (Micronized) | 0.011% | w/w; |
| Citric Acid Anhydrous (Micronized) | 1% | w/w; |
| Sodium Lauryl Sulfate | 6% | w/w; |
| Povidone K-25 | 6.5% | w/w; |
| Microcrystalline Cellulose | 23% | w/w; |
| Anhydrous Lactose | 50% | w/w; |
| Croscarmellose Sodium | 6% | w/w; |
| Colloidal Silicone Dioxide | 0.25% | w/w; and |
| Magnesium Stearate | 0.50% | w/w. |

Optionally, the tablets are film-coated. Suitable film-coatings are known to those of skill in the art. For example, the film-coating can be selected from among suitable polymers such as hydroxypropylmethylcellulose, ethyl cellulose, polyvinyl alcohol, and combinations thereof. Such coatings may also contain placticizers and other desirable components. In one embodiment, the coatings are inert. Other suitable film-coatings can be readily selected by one of skill in the art. Where applied, the weight percent of the film coat is generally in the range of 1% w/w to 6% w/w, about 2% w/w, about 3% w/w, about 4% w/w or about 5% w/w, and more desirably, about 2% w/w, based on the total weight of the coated composition.

The invention further provides a method of delivering CCI-779 to a patient, said method comprising the step of administering a micronized CCI-779 dosing unit according to the invention.

It is contemplated that when the formulations of this invention are used as an immunosuppressive or anti-inflammatory agent, they can be administered in conjunction with one or more other immunoregulatory agents. Such other antirejection chemotherapeutic agents include, but are not limited to azathioprine, corticosteroids, such as prednisone and methylprednisolone, cyclophosphamide, cyclosporin A, FK-506, OKT-3, and ATG. By combining one or more of the formulations of the present invention with such other drugs or agents for inducing immunosuppression or treating inflammatory conditions, lesser amounts of each of the agents may be required to achieve the desired effect. See, e.g., Transplantation Proc. 23: 507 (1991).

The dosage requirements may vary the severity of the symptoms presented and the particular subject being treated. Daily oral dosages of micronized CCI-779 can be about 0.05 to about 200 mg, about 0.05 to about 30 mg, about 5 mg to about 100 mg, about 10 mg to about 100 mg, about 1 mg to about 5 mg, about 1 mg to about 10 mg, about 1 mg to about 25 mg, about 1 mg to about 35 mg, about 1 mg to about 50 mg, about 20 mg to about 50 mg, about 5 mg to about 35 mg, about 25 mg to about 35 mg, about 25 mg to about 30 mg, about 5 mg, about 10 mg, about 15 mg, about 20 mg, about 25 mg, about 30 mg, or about 35 mg. In one example, when micronized CCI-779 is used in combination therapy at daily doses in the range of 0.5 to 10 mg. In another example, micronized CCI-779 is used in monotherapy at daily doses in the range of 1 mg to 30 mg. In other embodiments, daily doses are 2 to 5 mg when micronized CCI-779 is used in combination therapy, and 5 to 15 mg when micronized CCI-779 is used as monotherapy.

Treatment can be initiated with small dosages less than the optimum dose of the compound. Thereafter the dosage is increased until the optimum effect under the circumstances is reached. Precise dosages will be determined by the administering physician based on experience with the individual subject treated. In general, the formulations of this invention are most desirably administered at a concentration that will generally afford effective results without causing any unacceptable harmful or deleterious side effects.

Thus, the present invention provides a method of treating systemic lupus erythematosus, pulmonary inflammation, insulin dependent diabetes mellitus, skin disorders, bowel disorders, smooth muscle cell proliferation, intimal thickening following vascular injury, adult T-cell leukemia/lymphoma, ocular inflammation, malignant carcinomas, cardiac inflammatory disease, anemia, rheumatoid arthritis, and/or multiple sclerosis by administering a composition of the invention to a subject. The invention further provides for the use of the composition in preparing a medicament or product for use in these therapies and treatment regimens.

In another embodiment, the present invention provides products containing the compositions of the invention.

Suitably, the compositions of the invention are formulated such that a patient receives a suitable amount of the active component, e.g., about 0.05 to about 200 mg, about 0.05 to about 30 mg, about 5 mg to about 100 mg, about 10 mg to about 100 mg, about 1 mg to about 5 mg, about 1 mg to about 10 mg, about 1 mg to about 25 mg, about 1 mg to about 35 mg, about 1 mg to about 50 mg, about 20 mg to about 50 mg, about 5 mg to about 35 mg, about 25 mg to about 35 mg, about 25 mg to about 30 mg, about 5 mg, about 10 mg, about 15 mg, about 20 mg, about 25 mg, about 30 mg, or about 35 mg. Preferably, the formulations are such that a suitable dose is delivered in a single dosage unit. These doses may be administered daily for a suitable period of time, e.g., 4 weeks to 8 weeks, but can be delivered for a shorter period of time, e.g., 3 days to 3 weeks, one week to 3 months, or over a longer period, e.g., over 6 months, or longer. These compositions can be delivered alone or in combination with an antacid or other suitable composition.

Suitably, the compositions of the invention can be filled in capsules or caplets.

In one embodiment, the compositions of the invention are packaged for use by the patient or his caregiver, e.g., in a pharmaceutical pack or kit. For example, the compositions can be packaged in a foil or other suitable package.

The following examples are illustrative of specific embodiments of the invention and are not a limitation on the present invention. The following provide representative examples of the formulations of this invention. These examples are illustrative only, and do not limit the invention.

### EXAMPLE 1

### Directly Compressible Tablet Formulations Prepared By Employing Micronized CCI-779 And Poloxamer As Surfactant

The table formulations for this example are manufactured according to the following protocol.

Pass the poloxarner 188, microcrystalline cellulose (Avicel PH-112) and a portion of anhydrous lactose through a screen and blend. Mill the blend containing poloxamer with the help of a Fitz mill and transfer it to a V-blender of suitable size.

Preblend a portion of anhydrous lactose with micronized butylated hydroxyanisole, butylated hydroxytoluene, EDTA calcium disodium, hydrous, and citric acid anhydrous. Then add CCI-779 to this preblend, mix and add to the V-blender.

Take a portion of anhydrous lactose, croscarmellose sodium, and colloidal silicon dioxide (Aerosil 200) and pass through a screen, blend and transfer it to V-blender. Pass the remaining anhydrous lactose through a screen and transfer it to V-blender. Close the lids and blend the material without activation of the intensifier bar. Pass magnesium stearate through a screen, premix with a weight equivalent portion of powder blend and transfer the lubricant premix to V-blender and blend without the activation of the intensifier bar. Compress the final blend using a tablet press equipped with suitable tooling.

Quantitative Composition of CCI-779 Tablets, 25 mg
Containing Poloxamer

| | Percent | Mg/ | |
|---|---|---|---|
| Ingredients: | wt/wt | tablet | Function |
| CCI-779, Micronized | 6.250 | 25.00 | Active |
| Butylated Hydroxyanisol, NF | 0.022 | 0.088 | Antioxidant |
| Butylated Hydroxytoluene, NF | 0.050 | 0.20 | Antioxidant |
| EDTA, calcium disodium hydrous, USP | 0.011 | 0.044 | Chelating |
| | | | agent |
| Citric acid, Anhydrous USP | 0.080 | 0.32 | pH modifier |
| Poloxamer 188, NF | 6.250 | 25.00 | Surfactant |
| Lactose Anhydrous, NF | 55.060 | 220.24 | Filler |
| Microcrystalline Cellulose, NF | 27.527 | 108.58 | Filler/Binder |
| (Avicel PH 112) | | | |
| Croscarmellose Sodium, NF | 4.000 | 16.00 | Disintegrant |
| Aerosil 200, NF | 0.250 | 1.00 | Glidant |
| Magnesium Stearate, NF | 0.500 | 2.00 | Lubricant |
| Total | 100 | 400 | |

### EXAMPLE 2

### Directly Compressible Tablet Formulations Prepared By Employing Micronized CCI-779, Sodium Lauryl Sulfate And Povidone

The tablet formulations for this example are manufactured using the following protocol.

Microcrystalline cellulose (Avicel PH-112) and povidone K-25 are passed through a screen and transferred to a V-blender of suitable size. Micronized CCI-779 is preblended with a portion of lactose anhydrous separately, then passed through a screen and added to the V-blender. Sodium lauryl sulfate, croscarmellose sodium, silicone dioxide and a portion of lactose anhydrous are passed through a screen and transferred to the V blender. The remaining lactose anhydrous is passed through a screen and transferred it to V-blender and the lids are closed. The material is blended without activation of intensifier bar. Magnesium stearate is passed through a screen, premixed with a weight equivalent portion of powder, blended from V-blender, transferred to the lubricant premix to V-blender and blended without activation of intensifier bar. The final blend is compressed using a tablet press with suitable tooling.

Quantitative Composition of CCI-779 Tablets, 25 mg
Containing Low level of Povidone

| | Percent | Mg/ | |
|---|---|---|---|
| Ingredients | wt/wt | tablet | Function |
| CCI-779, Micronized | 6.250 | 25.00 | Active |
| Sodium Lauryl Sulfate, NF | 5.625 | 22.50 | Surfactant |
| Povidone, USP K25 | 6.250 | 25.00 | Filler/Binder |
| Lactose Anhydrous, NF | 50.583 | 202.33 | Filler |
| Microcrystalline Cellulose, NF | 24.543 | 98.172 | Filler/Binder |
| (Avicel PH 112) | | | |
| Croscarmellose Sodium, NF | 6.000 | 24.00 | Disintegrant |
| Aerosil 200, NF | 0.250 | 1.00 | Glidant |
| Magnesium Stearate, NF | 0.500 | 2.00 | Lubricant |
| Total | 100 | 400 | |

Quantitative Composition of CCI-779 Tablets, 25 mg
Containing High Level of Povidone

| | Percent | Mg/ | |
|---|---|---|---|
| Ingredients: | wt/wt | tablet | Function |
| CCI-779, Micronized | 6.250 | 25.00 | Active |
| Sodium Lauryl Sulfate, NF | 5.625 | 22.50 | Surfactant |
| Povidone, USP K-25 | 31.250 | 125.00 | Filler/Binder |
| Lactose Anhydrous, NF | 33.750 | 135.00 | Filler |
| Microcrystalline Cellulose, NF | 16.375 | 65.50 | Filler/Binder |
| (Avicel PH 112) | | | |
| Croscarmellose Sodium, NF | 6.000 | 24.00 | Disintegrant |
| Silicone dioxide (Aerosil 200), NF | 0.250 | 1.00 | Glidant |
| Magnesium Stearate, NF | 0.500 | 2.00 | Lubricant |
| Total | 100 | 400 | |

### EXAMPLE 3

### Bioavailability Study

A. Number Of Patients:

A total of 40 subjects, 38 men and 2 women, were enrolled in the study, and 35 subjects, 33 men and 2 women, completed the study (40 planned, 40 enrolled, 35 completed, 40 analyzed for safety, 35 analyzed for pharmacokinetics).

B. Duration Of Treatment:

Each completing subject participated in study procedures for approximately 43.5 days. The prestudy screening evaluation took place within 2 to 14 days before study day 1 of period 1.

C. Study Drug, Dose, And Mode Of Administration:

On day 1 of each of the 3 study periods, the subjects received a single oral dose of 1 of 4 treatments (treatments A, B, C, or reference).

Treatment A was CCI-779 25 mg tablet, containing micronized Poloxamer 188, manufactured essentially as described in Example 1. For the reasons illustrated below (including lower bioavailability), this Treatment is less desirable than prototypes B and C described herein.

Test treatment B was CCI-779 25 mg tablet (Micronized High Povidone), manufactured essentially as described in Example 2.

Test treatment C was CCI-779 25 mg tablet (Micronized Povidone API [active pharmaceutical] Quantity), manufactured essentially as described in Example 2.

Reference treatment D was 25 mg CCI-779 consisting of CCI-779 10 mg tablet, manufactured by Wyeth Pharmaceuticals, Inc.; and CCI-779 5 mg tablet, manufactured by Wyeth Pharmaceuticals, Inc.

D. Dose And Mode Of Administration:

On day 1 of each of the 3 study periods, the subjects received a single oral dose of 1 of 4 treatments (A, B, C, or reference).

E. Pharmacokinetics/Pharmacodynamic And Statistical Methods:

The following pharmacokinetic parameters for CCI-779 and sirolimus, which is the major metabolite of CCI-779 in whole blood, were calculated from whole blood concentration-time data using noncompartmental methods: AUCₒ₋ₜ, AUC, AUCR [AUC₀₋ₜ/AUC], K_{cl}, t_{1/2}, Cₘₐₓ, and tₘₐₓ. CL/F and V_{z}/F were calculated for CCI-779. In addition, the AUCᵣₐₜᵢₒ [the ratio of sirolimus AUC:CCI-779 AUC] and AUCₛᵤₘ [the sum of sirolimus AUC and CCI-779 AUC] were calculated.

A parametric (normal theory) general linear model was applied to the log-transformed AUC, AUC₀₋ₜ, and Cₘₐₓ values for each analyte. The 90% confidence interval (CI) for the ratio of the test and reference geometric least squares (LS) means (A vs D, B vs D, and C vs D) was determined for each parameter.

If the 90% confidence intervals (CI) for the ratios of the test and reference geometric LS means for all 3 parameters included 100%, then it was concluded that there was no significant difference in bioavailability between the test and reference formulations. A higher bioavailability for the test formulation was concluded if the lower limits of the 90% CI were larger than 100%, and a lower bioavailability was concluded if the upper limits of the 90% CI were smaller that 100%.

CCI-779 prototype A -
Pharmacokinetics based on CCI-779

| | Whole Blood CCI-779 | | | | | | | |
|---|---|---|---|---|---|---|---|---|
| | Treatment A^{a} | | | Treatment D^{b} | | | | |
| Pharmacokinetic | | Arithmetic | | | Arithmetic | | | % Mean |
| Parameters | N | Mean | SD | N | Mean | SD | 90% CI^{c} | Ratio^{c} |
| Cₘₐₓ (ng/mL) | 25 | 2.912 | 0.820 | 26 | 7.925 | 3.615 | — | — |
| Tₘₐₓ (hr)^{d} | 25 | 4.12 | 4.61 | 26 | 1.15 | 0.368 | — | — |
| AUC₀₋ₜ | 25 | 46.12 | 20.27 | 26 | 58.43 | 24.91 | — | — |
| (ng * hr/mL) | | | | | | | | |
| AUC (ng * hr/mL) | 20 | 60.1 | 17.77 | 26 | 69.64 | 23.90 | — | — |
| ln(Cₘₐₓ) | 25 | 1.030 | 0.2888 | 26 | 1.995 | 0.3765 | 34.88-45.97 | 40.0 |
| ln[AUC₀₋ₜ] | 25 | 3.752 | 0.4001 | 26 | 3.996 | 0.3730 | 69.73-91.72 | 80.0 |
| ln[AUC] | 20 | 4.054 | 0.3006 | 26 | 4.193 | 0.3188 | 79.34-100.6 | 89.3 |

^{a}Treatment A = 1 × 25 mg CCI-779 tablet (micronized poloxamer): test
^{b}Treatment D = 2 × 10 mg plus 1 × 5 mg CCI-779 tablet: reference
^{c}Values calculated using LS Means.
^{d}The median values for Tₘₐₓ were 3 hours (treatment A) and 1 hour (treatment D).

CCI-779 prototype B
Pharmacokinetics based on CCI-779

| | Whole Blood CCI-779 | | | | | | | |
|---|---|---|---|---|---|---|---|---|
| | Treatment B^{a} | | | Treatment D^{b} | | | | |
| Pharmacokinetic | | Arithmetic | | | Arithmetic | | | % Mean |
| Parameters | N | Mean | SD | N | Mean | SD | 90% CI^{c} | Ratio^{c} |
| Cₘₐₓ (ng/mL) | 29 | 5.419 | 1.811 | 26 | 7.925 | 3.615 | — | — |
| Tₘₐₓ (hr)^{d} | 29 | 2.00 | 1.77 | 26 | 1.15 | 0.368 | — | — |
| AUC₀₋ₜ | 29 | 50.67 | 21.79 | 26 | 58.43 | 24.91 | — | — |
| (ng * hr/mL) | | | | | | | | |
| AUC | 24 | 66.36 | 21.91 | 26 | 69.64 | 23.90 | — | — |
| (ng * hr/mL) | | | | | | | | |
| Ln(Cₘₐₓ) | 29 | 1.641 | 0.3130 | 26 | 1.995 | 0.3765 | 65.22-84.52 | 74.2 |
| Ln[AUC₀₋ₜ] | 29 | 3.838 | 0.4301 | 26 | 3.996 | 0.3730 | 82.36-106.54 | 93.7 |
| Ln[AUC] | 24 | 4.142 | 0.3388 | 26 | 4.193 | 0.3188 | 89.65-111.37 | 99.9 |

^{a}Treatment B = 1 × 25 mg CCI-779 tablet (high povidone): test
^{b}Treatment D = 2 × 10 mg plus 1 × 5 mg CCI-779 tablet: reference
^{c}Values calculated using LS Means
^{d}The median values for Tₘₐₓ were 2 hours (treatment B) and 1 hour (treatment D).

CCI-779 prototype C for CCI-779
Pharmacokinetics of CCI-779

| | Whole Blood CCI-779 | | | | | | | |
|---|---|---|---|---|---|---|---|---|
| | Treatment C^{a} | | | Treatment D^{b} | | | | |
| Pharmacokinetic | | Arithmetic | | | Arithmetic | | | % Mean |
| Parameters | N | Mean | SD | N | Mean | SD | 90% CI^{c} | Ratio^{c} |
| Cₘₐₓ (ng/mL) | 25 | 5.727 | 1.668 | 26 | 7.925 | 3.615 | — | — |
| Tₘₐₓ (hr)^{d} | 25 | 1.32 | 0.627 | 26 | 1.15 | 0.368 | — | — |
| AUC₀₋ₜ | 25 | 51.92 | 20.10 | 26 | 58.43 | 24.91 | — | — |
| (ng * hr/mL) | | | | | | | | |
| AUC | 23 | 59.67 | 20.43 | 26 | 69.64 | 23.90 | — | — |
| (ng * hr/mL) | | | | | | | | |
| In(Cₘₐₓ) | 25 | 1.703 | 0.3024 | 26 | 1.995 | 0.3765 | 71.41-94.12 | 82.0 |
| In[AUC₀₋ₜ] | 25 | 3.881 | 0.3772 | 26 | 3.996 | 0.3730 | 85.04-111.87 | 97.5 |
| In[AUC] | 23 | 4.039 | 0.3158 | 26 | 4.193 | 0.3188 | 85.60-106.99 | 95.7 |

^{a}Treatment C = 1 × 25 mg CCI-779 tablet (low povidone API quantity): test
^{b}Treatment D = 2 × 10 mg plus 1 × 5 mg CCI-779 tablet: reference
^{c}Values calculated using LS Means
^{d}The median values for Tₘₐₓ were 1 hour (treatment C) and 1 hour (treatment D).

CCI-779 prototype A for sirolimus (rapamycin)

| | Whole Blood Sirolimus | | | | | | | |
|---|---|---|---|---|---|---|---|---|
| | Treatment A | | | Treatment D | | | | |
| Pharmacokinetic | | Arithmetic | | | Arithmetic | | | % Mean |
| Parameters | N | Mean | SD | N | Mean | SD | 90% CI* | Ratio* |
| Cₘₐₓ (ng/mL) | 25 | 11.358 | 6.986 | 26 | 27.458 | 12.404 | — | — |
| Tₘₐₓ (hr)^{d} | 25 | 4.08 | 2.12 | 26 | 1.39 | 0.637 | — | — |
| AUC₀₋ₜ | 25 | 483.0 | 139.6 | 26 | 595.5 | 189.1 | — | — |
| (ng * hr/mL) | | | | | | | | |
| AUC | 25 | 538.8 | 149.4 | 26 | 664.1 | 217.5 | — | — |
| (ng * hr/mL) | | | | | | | | |
| In(Cₘₐₓ) | 25 | 2.324 | 0.4226 | 26 | 3.218 | 0.4429 | 35.06-47.00 | 40.6 |
| In[AUC₀₋ₜ] | 25 | 6.142 | 0.2773 | 26 | 6.343 | 0.3097 | 72.82-88.54 | 80.3 |
| In[AUC] | 25 | 6.254 | 0.2674 | 26 | 6.448 | 0.3224 | 73.59-88.84 | 80.9 |

Treatment A = 1 × 25 mg CCI-779 Tablet (Micronized Poloxamer): test
Treatment D = 2 × 10 mg Plus 1 × 5 mg CCI-779 Tablet: reference
**= The median values for Tₘₐₓ were 3 hours (treatment A) and 1 hour (treatment D).
*= Values calculated using LS Means

CCI-779 prototype B for sirolimus (rapamycin)

| | Whole Blood Sirolimus | | | | | | | |
|---|---|---|---|---|---|---|---|---|
| | Treatment B | | | Treatment D | | | | |
| Pharmacokinetic | | Arithmetic | | | Arithmetic | | | % Mean |
| Parameters | N | Mean | SD | N | Mean | SD | 90% CI* | Ratio* |
| Cₘₐₓ (ng/mL) | 29 | 18.697 | 9.568 | 26 | 27.458 | 12.404 | — | — |
| Tₘₐₓ (hr)^{d} | 29 | 2.86 | 2.10 | 26 | 1.39 | 0.637 | — | — |
| AUC₀₋ₜ | 29 | 537.4 | 211.9 | 26 | 595.5 | 189.1 | — | — |
| (ng * hr/mL) | | | | | | | | |
| AUC | 29 | 599.8 | 228.2 | 26 | 664.1 | 217.5 | — | — |
| (ng * hr/mL) | | | | | | | | |
| Ln(Cₘₐₓ) | 29 | 2.830 | 0.4274 | 26 | 3.218 | 0.4429 | 60.24-79.33 | 69.1 |
| Ln[AUC₀₋ₜ] | 29 | 6.221 | 0.3635 | 26 | 6.343 | 0.3097 | 84.38-101.38 | 92.5 |
| Ln[AUC] | 29 | 6.335 | 0.3545 | 26 | 6.448 | 0.3224 | 85.22-101.70 | 93.1 |

Treatment B = 1 × 25 mg CCI-779 Tablet (High Povidone): test
Treatment D = 2 × 10 mg Plus 1 × 5 mg CCI-779 Tablet: reference
**= The median values for Tₘₐₓ were 2 hours (treatment B) and 1 hour (treatment D).
*= Values calculated using LS Means

CCI-779 prototype C for sirolimus

| | Whole Blood Sirolimus | | | | | | | |
|---|---|---|---|---|---|---|---|---|
| | Treatment C | | | Treatment D | | | | |
| Pharmacokinetic | | Arithmetic | | | Arithmetic | | | % Mean |
| Parameters | N | Mean | SD | N | Mean | SD | 90% CI* | Ratio* |
| Cₘₐₓ (ng/mL) | 25 | 17.064 | 8.070 | 26 | 27.458 | 12.404 | — | — |
| Tₘₐₓ (hr)^{d} | 25 | 2.16 | 0.898 | 26 | 1.39 | 0.637 | — | — |
| AUC₀₋ₜ | 25 | 491.7 | 171.4 | 26 | 595.5 | 189.1 | — | — |
| (ng * hr/mL) | | | | | | | | |
| AUC | 25 | 547.6 | 187.0 | 26 | 664.1 | 217.5 | — | — |
| (ng * hr/mL) | | | | | | | | |
| Ln(Cₘₐₓ) | 25 | 2.749 | 0.4185 | 26 | 3.218 | 0.4429 | 60.20-80.72 | 69.7 |
| Ln[AUC₀₋ₜ] | 25 | 6.136 | 0.3748 | 26 | 6.343 | 0.3097 | 80.92-98.40 | 89.2 |
| Ln[AUC] | 25 | 6.246 | 0.3671 | 26 | 6.448 | 0.3224 | 81.56-98.45 | 89.6 |

Treatment C = 1 × 25 mg CCI-779 Tablet (low povidone): test
Treatment D = 2 × 10 mg Plus 1 × 5 mg CCI-779 Tablet: reference
**= The median values for Tₘₐₓ were 2 hours (treatment C) and 1 hour (treatment D).
*= Values calculated using LS Means

F. Results:

Of the 40 subjects who received CCI-779, 19 subjects (48%) had at least 1 treatment-emergent adverse event (AE). Fewer subjects reported AEs after the CCI-779 prototype formulations (treatments A, B, and C) compared with the CCI-779 reference formulation (treatment D): 3 of 26 subjects (12%) reported AEs following treatment A, 3 of 29 subjects (10%) reported AEs following treatment B, 5 of 29 subjects (17%) reported AEs following treatment C, and 12 of 29 subjects (41%) reported AEs following treatment D.

All 45 treatment-emergent AEs were mild in severity. The investigator considered 16 of the 45 AEs to be related to CCI-779 treatment. No deaths or other serious adverse events occurred during this study. Following administration of treatment D, 1 subject was discontinued from the study because the AEs of lung disorder (verbatim term “chest congestion”), pharyngitis, and rhinitis which the investigator considered to be unrelated to study treatment. Following treatment D administration in period 2, 2 subjects were withdrawn at period 3 check-in because of laboratory abnormalities including elevated aminotransferase AEs that the investigator considered to be study-drug related. Following treatment C administration in period 1, 1 subject was withdrawn at period 2 check-in because of urinalysis abnormalities including the AE of hematuria, which the investigator considered to be unrelated to study drug.

A total of 13 subjects had clinically significant laboratory abnormalities after dose administration. The most common clinically significant laboratory abnormalities included white blood cells in the urine, that occurred in a total of 5 subjects (13%), and aminotransferase elevations, occurred in a total of 4 subjects (10%). Clinically significant abnormalities in all remaining laboratory parameters were isolated instances had by fewer than 10% of subjects. Most clinically significant laboratory abnormalities present after 1 of the test CCI-779 treatments also occurred following the reference CCI-779 treatment. Thus, no treatment-related trends were noted. Two (2) subjects had aminotransferase elevations considered to be AEs and 3 subjects (including the 2 subjects with transaminase AEs) were dropped from the study because of laboratory abnormalities. Despite these individual laboratory abnormalities, all serum chemistry and hematology mean values remained within their respective reference ranges.

No clinically significant trends were noted in the vital sign measurements, electrocardiogram results, or physical examination findings.

G. Conclusions:

Average bioavailability of CCI-779 and exposure of sirolimus from the micronized poloxamer tablet (treatment A) was lower than the micronized reference formulation (treatment D). AUC and Cₘₐₓ were 11% to 20% and 60% lower, respectively. With the exception of CCI-779 AUC, the upper limits of the 90% confidence intervals for the Cₘₐₓ, AUC₀₋ₜ, and AUC ratios were all less than 100%. Median tllax for treatment A was 2 hours later than treatment D. The lower Cₘₐₓ and later tₘₐₓ indicate that the rate of absorption from the micronized poloxamer formulation differed from the reference formulation.

There was no difference in AUC for CCI-779 and sirolimus between the micronized high povidone tablet (treatment B) and the reference formulation. Geometric LS mean Cₘₐₓ values for the high povidone tablet were 26% to 31% lower than for the reference formulation. Median tₘₐₓ was also 1 hour later for treatment B, which may indicate a difference in the rate of absorption between the 2 formulations.

There was no difference in AUC for CCI-779 from the low povidone tablet (treatment C) and the reference formulation. CCI-779 Cₘₐₓ was slightly lower (18%) for treatment C whereas median tₘₐₓ values were equivalent between treatment C and the reference formulation. The exposure of sirolimus from treatment C was slightly lower than from the reference formulation, with differences of 10% to 11% (AUC) and 30% (Cₘₐₓ) in the geometric LS means and 90% confidence intervals not inclusive of 100%. Median sirolimus tₘₐₓ was 1 hour later for treatment C.

Prototype formulations of CCI-779 (Micronized Poloxamer 188, High Povidone, and Povidone API Quantity) appeared to be as safe and well tolerated by the healthy male and female subjects in this study as the single reference formulation of CCI-779 when administered in single 25 mg doses.

The documents listed throughout this specification are hereby incorporated by reference. Minor variations and modifications to the methods and materials set forth in the foregoing detailed description and illustrative examples will be readily apparent to those of skill in the art and are encompassed within the scope of the invention.

1. A composition comprising an oral dosage form containing an effective amount of CCI-779 wherein, after oral administration thereof to a subject, the CCI-779 has a Cₘₐₓ of 5.4±1.8 ng/mL and an AUC of about 66±about 22 ng-hr/ml and a sirolimus Cₘₐₓ of 18.7±9.6 ng/mL and an AUC of about 600±about 228 ng-hr/ml in whole blood, for a 25 mg unit dose of CCI-779.

2. The composition according to claim 1, further characterized by having a CCI-779 Tₘₐₓ of 2.0±1.8 hours.

3. A composition comprising an oral dosage form containing an effective amount of CCI-779 wherein, after oral administration thereof to a subject, the CCI-779 has a Cₘₐₓ of 5.7±1.7 ng/mL and an AUC of about 60±about 20 ng-hr/ml and a sirolimus Cₘₐₓ of 17.1±8.1 ng/mL and an AUC of about 548±about 187 ng-hr/ml in whole blood, for a 25 mg unit dose of CCI-779.

4. The composition according to claim 3, further characterized by having a CCI-779 Tₘₐₓ of 1.3±0.6 hours.

5. The composition according to claim 1, wherein said oral dosage form is a tablet.

6. The composition according to claim 1, wherein said oral dosage form contains 5 to 35 mg CCI-779.

7. The composition according to claim 6, wherein said oral dosage form contains 25 mg CCI-779.

8. The composition according to claim 6, wherein said oral dosage form contains 30 mg CCI-779.

9. A method of treating systemic lupus erythematosus, pulmonary inflammation, insulin dependent diabetes mellitus, skin disorders, bowel disorders, smooth muscle cell proliferation, intimal thickening following vascular injury, adult T-cell leukemia/lymphoma, ocular inflammation, malignant carcinomas, cardiac inflammatory disease, anemia, rheumatoid arthritis, and multiple sclerosis by administering a composition according to claim 1 to a subject.

10. The method according to claim 9, wherein a tablet comprising 25 mg is delivered to the subject.

11. The method according to claim 9, wherein a tablet comprising 30 mg is delivered to the subject.

12. The method according to claim 9, wherein multiple oral dosage forms are delivered to the subject.

13. The method according to claim 9, wherein each of the multiple oral dosage forms contain from 5 mg to 35 mg CCI-779.

14. A pharmaceutical pack comprising a composition according to claim 1 and packaging for said composition.
